(19) United States
(12) Patent Application Publication
(10) Pub. No.: US 2024/0110912 A1
(43) Pub. Date: Apr. 4, 2024

(54) DIAGNOSTIC TESTING

(71) Applicant: PARAYTEC LTD, Osbalawick York Yorkshire (GB)

(72) Inventors: Carl Glenton Weymouth SMYTHE, Sheffield Yorkshire (GB); David Murray GOODALL, Huntington York Yorkshire (GB)

(21) Appl. No.: 18/265,008

(22) PCT Filed: Jan. 14, 2021
(86) PCT No.: PCT/GB2021/050083

§ 371 Date: Jun. 2, 2023

(30) Foreign Application Priority Data
Dec. 2, 2020 (GB) 2019024.5

(51) Int. Cl. G01N 33/569; G01N 33/533

(52) U.S. Cl. CPC G01N 33/569; G01N 33/533

(57) ABSTRACT

The present invention concerns the field of diagnostic testing to confirm the presence of target pathogens in a test sample, typically obtained from a human or animal. The test has particular application in testing for viruses, but could include cancer cells or other particulate pathogens, including bacteria. The disclosure provides a method for facilitating the detection of target pathogen particles in a test sample, the method comprising: providing one or more supports defining a support surface, the support surface being provided with a coating of a first set of macromolecular assemblies, the assemblies each being capable of selectively binding with the target pathogen particles, providing a second set of macromolecular assemblies, the assemblies also each being capable of binding selectively with the target pathogen particles, and wherein each of the second set of macromolecular assemblies is provided with at least one fluorophore moiety, obtaining or providing the test sample to be assayed for the presence of the target pathogen therein, exposing the macromolecular assemblies to the test sample so that target pathogen particles present in the test sample bind to the macromolecular assemblies, thereby producing a multitude of target pathogen particles distributed over and anchored to said support surface by members of the first set of macromolecular assemblies, with a plurality of fluorophore moieties being bound to each pathogen particle by members of the second set of macromolecular assemblies, so as to produce a fluorophore coating on anchored pathogen particles on the support. The invention provides assemblies of pathogen particles produced by this method. The methods may include a detection step for detecting fluorescence from bound fluorophores.

The present invention concerns the field of diagnostic testing to confirm the presence of target pathogens in a test sample, typically obtained from a human or animal. The tests have particular application in testing for viruses, but could include cancer cells or other particulate pathogens, including bacteria. The invention also concerns assemblies of fluorescently labelled pathogen particles, and kits for capturing pathogens from test samples and labelling them to permit detection.

In testing for viruses, we are concerned in particular with testing to establish the presence of a target virus in a test sample, and any associated quantitative information that can be measured. Recent years outbreaks of severe acute respiratory syndrome coronavirus (SARS-CoV) in 2003 and Middle East Respiratory Syndrome Coronavirus (MERS-CoV) in 2012 resulted in death rates of 10% and 35% respectively (Canrong Wu et al, Acta Pharmaceutica Sinica B, Vol. 10, 5, May 2020 pp 766-788). More recently the development of a pandemic caused by SARS-CoV-2 and its associated disease COVID-19 has resulted to date in the reported deaths of 1.43 million people (https://ourworldindata.org/covid-deaths, inspected 27 Nov. 2020).

Management and suppression of the pandemic relies upon the wide availability of diagnostic tests to establish whether a person is currently infected, irrespective of whether symptoms are evident. More generally there is a need for a test which can rapidly and accurately screen populations for infection, whether symptomatic or not, so that targeted quarantines or isolation can be implemented, and precautionary isolations reduced or avoided.

Currently, most tests for COVID-19 rely upon PCR amplification of viral genetic matter in test samples (typically saliva or throat swabs) to indicate the presence of SARS-CoV-2 RNA, thereby confirming infection with COVID-19, in line with WHO recommendations (WHO Reference No. WHO/2019-nCoV/SciBrief/POC immunodiagnostics/2020.1)

There is a multitude of diagnostic tests in use and, in the USA alone, some 180 emergency use authorisations (EUAs) have been granted by the FDA for diagnostic tests for COVID-19. These all rely upon RT-PCR amplification methods for the virus' genetic material obtained from respiratory samples (e.g. sputum or throat swab). Antigen tests are also in use which confirm the presence of viral antigens but cannot confirm a current live infection.

It is known to use a DNA aptamer-based method to detect SARS-CoV-2 by targeting the nucleocapsid protein (N protein). This is said to be capable of very early detection of COVID as the protein can be detected as early as one day after onset of the disease (Chen et al, Virologica Sinica 35, 351-354 (25 May 2020). In this method, N protein is coated onto plates, the N protein is detected using biotinylated ssDNA aptamers followed by a horseradish peroxidase conjugated avidin system.

Antibody tests have also been developed to establish whether a person has been exposed to coronavirus. This is usually done by taking a blood sample and testing for antibodies developed in response to the virus. These cannot differentiate between active infections and past infections, so cannot be used to test if a person currently has COVID-19.

One emergency use authorisation (EUA) has been granted for a microfluidic immunofluorescence assay in which nasal swabs are used to gather saliva from individuals suspected of having contracted COVID-19. Qualitative detection of the nucleocapsid protein antigen from SARS-CoV-2 is made directly using a portable microfluidic diagnostic apparatus.

The present invention seeks in one aspect to provide a general method for facilitating the detection of particulate pathogens such as bacteria, cancer cells or viruses for diagnostic purposes.

In another aspect the invention seeks to provide a testing method which avoids the need for PCR amplification, which involves time-consuming thermal cycling and enzyme-driven DNA replication.

In yet another aspect the invention aims to provide a pathogen test which provide not just a qualitative indication of live infection, but also quantitative information on the amount of virus present.

The invention also seeks to provide a detection kit with components which capture pathogen particles and label them to facilitate detection.

In an ancillary aspect the invention seeks to provide a virus mimic so that use of live virus may be avoided or limited in development of the testing methods of this invention or others.

According to one aspect of the invention there is provided a method for facilitating the detection of target pathogen particles in a test sample, the method comprising:
providing one or more support defining a support surface, the support surface being provided with a coating of a first set of macromolecular assemblies, the assemblies each being capable of selectively binding with the target pathogen particles,
providing a second set of macromolecular assemblies, the assemblies also each being capable of binding selectively with the target pathogen particles, and wherein each of the second set of macromolecular assemblies is provided with at least one fluorophore moiety, obtaining or providing the test sample to be assayed for the presence of the target pathogen therein,
exposing the macromolecular assemblies to the test sample so that target pathogen particles present in the test sample bind to the macromolecular assemblies, thereby producing a multitude of target pathogen particles distributed over and anchored to said support surface by members of the first set of macromolecular assemblies, with a plurality of fluorophore moieties being bound to each pathogen particle by members of the second set of macromolecular assemblies, so as to produce a fluorophore coating on anchored pathogen particles on the support.

The method may involve exposing the test sample to the sets of macromolecular assemblies sequenced so that the first and second sets of macromolecular assemblies are exposed to the test sample concurrently, or one before the other. In a preferred sequence the first set is exposed to the test sample before the second set of macromolecular assemblies, so that the anchorage of the pathogen particles to the support takes place substantially before the binding of the assemblies provided with the fluorophore moieties to the pathogen particles.

The said at least one support may comprise one or more selected from: a plurality of support particles, or a plurality of fibres, or a felt material, or a mesh, or a grid, or a frit, or a chromatography monolith, or a textile or a filter material, or other porous membrane. The material from which the support is made will typically be a solid, whatever its form configuration.

In another aspect of the invention, there is provided a diagnostic method for detecting target pathogen particles in a test sample comprising conducting the method hereinbefore described and further comprising exposing the fluorophore-coated support to incident light radiation so as to cause excitation of the fluorophores and emission of fluorescence.

The method typically further comprises detecting the emitted fluorescence so as to indicate the presence of pathogen particles in the test sample. The fluorescence may be detected by fluorescence microscopy. The method may comprise imaging individual fluorophore coated support particles by detecting emitted fluorescence from each one. The detected fluorescence in the field of view can be integrated to provide a quantitative measure that can be correlated to the amount of pathogen present.

In a preferred aspect the support may comprise a particulate material and the detection may be by flow cytometry apparatus in which support particles are induced to flow sequentially through a region illuminated by light from a focused excitation source, and emitted fluorescence is detected from each fluorophore coated support particle. The cytometric apparatus is typically adapted to determine the intensity of fluorescence of each fluorophore-coated support particle. The intensity of emission detected may be accumulated/integrated and correlated to the amount of pathogen present in the sample.

Hence both qualitative and/or quantitative information can be obtained during the detection step.

In general, the test sample is provided in the form of a liquid with pathogen particles dispersed therein. The liquid may comprise saliva, mucous or other body fluids obtained from a person or animal which may contain the target pathogen. These may be conveniently obtained via the use of a swab. For a respiratory infection, the liquid may be obtained from a nasopharyngeal swab sample. The liquid may comprise serum or blood if these contain the target pathogen.

Alternatively a sample from a patient can be provided as a breath sample, so could comprise an aerosol of liquid droplets each having pathogen particles dispersed therein. The aerosol may be passed through a reception liquid to deposit the pathogen particles therein so as to produce a liquid test sample for subsequent analysis.

The step of obtaining a sample from a person or animal may or may not from part of the method steps of the present invention. The sample may be provided having been previously obtained by conventional methods, such as the use of a swab. The sample may have been purified or diluted or buffered or otherwise treated to improve its suitability for analysis in the present method. In a swab test a swab with the saliva or mucous is placed in an aqueous medium. This extracts the target pathogen when the swab is pressed against the surface of the medium container. This aqueous medium is then the test sample.

The liquid test sample may wet the support by capillary action, or be induced to flow over the support surface, such as by pumping or gravity or suction.

The support may be arranged to be immobile with respect to an exposed liquid test sample. There may be an induced flow of test sample over the macromolecular assembly coated on the support so as to expose the coating to pathogen particles. So the test sample may be induced to flow through a bed of coated support particles, or a coated mesh support or a coated group of fibres, or another of the supports hereinbefore mentioned (after coating with the first set of macromolecular assemblies).

Thus in a further aspect of the invention the support comprises support particles coated with the first set of assemblies and arranged as a bed of particles, or a packed conglomeration of particles. This is then exposed to the liquid test sample.

For example, a fluid comprising the test sample may be introduced to the coated support particles, so that any pathogen particles present bind to the coated support particles. A liquid comprising the second set of assemblies provided with fluorophores may be introduced to the support particles so that fluorophores become associated with pathogen particles, whether free pathogen particles or those anchored to the support particles.

The support may in a preferred aspect comprise support particles comprising microspheres, or a granular material. Microspheres are particularly preferred as they are commercially available in a range of micron grades, and in some cases functionalised with carboxyl or amide groups to facilitate derivatisation. The support particles may have a diameter of about 0.5 to 100 microns, preferably about 0.5 to 20 microns.

The size of the particles (especially microspheres) may be selected with reference to the target pathogen, so that larger particles may suit larger pathogens, in order to ensure space for multiple pathogens to become anchored thereon, and a strong fluorescence signal is enabled for each particle. Substantially uniform support particle sizes are preferred. Microspheres are commercially available in a range of specific diameters. Particulate materials having a size distribution can if necessary be graded to a substantially uniform size by sieving or filtering.

So in yet another preferred aspect of the invention a plurality of pathogen particles become bound to each support particle and a multitude of fluorophore moieties are bound to each pathogen particle. In the context of this patent application a multitude means more than a plurality, for example, an order of magnitude more.

Turning to the signalling aspect of the invention, each fluorophore moiety may comprise a fluorescent molecule or group of fluorescent molecules. In preferred embodiments the fluorophore moiety comprises a fluorescent nanobead. Each nanobead will typically comprise a multitude of fluorescent molecules, such as fluorescein. In a preferred example there are more than 100 fluorescent molecules per nanobead. Fluorescent nanobeads are available commercially in the form of fluorescein (or the like) molecules blended or mixed with a clear polymer such as polystyrene.

By nanobead we mean a bead (or sphere) which has at least one dimension (usually diameter) that is 100 nm or less. The bead is typically formed of a clear polymer material, especially polystyrene.

The macromolecular assembles typically each comprise or consist of proteins and/or nucleic acids. The macromolecular assemblies may comprise an aptamer for the pathogen, or a receptor for the pathogen. In some embodiments, one set of macromolecular assemblies comprises aptamers and the other set comprises receptors for the target pathogen. The second set of macromolecular assemblies may comprise chimeric proteins, one portion of which is adapted to bind to the pathogen and the other portion of which is bound to the fluorophore moiety.

The production of aptamers is well known by the Systematic Evolution of Ligands by Exponential Enrichment (SELEX) in vitro selection method. Pre-selected aptamers for certain pathogens may be obtained commercially. Virus receptors represent surface proteins on cells which virus surface proteins may target to infect the cell. As such they can when isolated be used to engage viral surface proteins, so as to bond therewith. A virus particle is not usually considered captured until there is binding between the viral surface proteins and the macromolecular assembly. Multiple such non-covalent binding events may be expected to increase the avidity.

Generally, the target pathogen particle may be a virion (an intact virus assembly), part of a virus, a bacterium, a cancer cell. Viruses are nanoscale particles, most of which have a diameter of about 20-300 nm. A preferred target is Corona viruses which are 65-125 nm diameter (Shereen et al, J.Adv.Res. Vol.24 Jul. 2020 pp91-98). The virion is a preferred target, as a live virus.

The pathogen is in some preferred applications of the invention a virion, a portion of a virion, a bacterium, or a cancer cell. The pathogen may be a corona virus, a corona virion, most preferably SARS-CoV-2 the cause of COVID-19.

The second set of macromolecular assemblies comprises a chimeric protein and one portion of the chimeric protein is selected from an aptamer for the virion, or a receptor for the virion, and the other portion is bound to the fluorophore moiety.

Each virion is bound to a support particle by an aptamer which binds conformally with the virion, or by a receptor which binds with a surface protein of the virion. In a preferred embodiment one portion of the chimeric protein comprises angiotensin-converting enzyme 2 (ACE2) which serves as a receptor for the coronavirus.

The fluorophore moiety bound to each chimeric protein may comprise one or more fluorescent nanobead. One portion of the chimeric protein may comprise angiotensin-converting enzyme 2 (ACE2) which serves as a receptor for the coronavirus. The chimeric protein may be ACE2-Fc, where Fc is the Fc region of any human immunoglobulin molecule. Fc has binding specificity for protein G. The nanobeads may be treated with Protein G so as to induce the attachment of the beads to the chimeric protein.

One or more binding agents are typically used to promote or effect binding between the support surface and the first set or macromolecular assemblies. Similarly, one or more binding agents are typically used to promote or effect binding between the fluorophore moieties and the second set of macromolecular assemblies.

In certain embodiments avidin and Protein G may be used as binding agents for binding the macromolecular assemblies to the microspheres or nanobeads.

The invention may provide a fluorescent assembly comprising a support with a plurality of pathogen particles anchored to the support by a first set of macromolecular assemblies, and a plurality of fluorophore moieties attached to each pathogen particle by a second set of macromolecular assemblies. This fluorescent assembly may be obtained by a method as hereinbefore described, omitting the detection step.

In yet a further preferred aspect the invention provides an assembly of pathogen particles comprising:
a support defining a support surface,
a first set of macromolecular assemblies each having one region bound to the support surface and another region bound to a target pathogen particle, so that there is a multitude of anchored target pathogen particles on the support surface,
a second set of macromolecular assemblies each having one region bound to the pathogen particles and another region with a fluorophore moiety attached thereto.

Preferably, each support has multiple pathogen particles attached thereto, and multiple fluorophore moieties attached to each pathogen particle.

The fluorophore moieties preferably comprise fluorescent nanobeads each comprising multiple fluorescent molecules.

In a further aspect useful of making a pathogen testing kit, there is provided a target pathogen particle labelling system comprising:
(i) a capture module comprising a support defining a support surface, and a first set of macromolecular assemblies each having one region bound to the support surface, and another region which is adapted to selectively bind with the target pathogen particle; and
(ii) a signal module comprising a second set of macromolecular assemblies each having one region adapted to selectively bind to pathogen particles, and another region with a fluorophore moiety attached thereto,
wherein a test sample containing comprising pathogen particles may be exposed to multiple capture modules and multiple signal modules so as to cause multiple pathogen particles to become bound to the support by the first set of assemblies, and multiple fluorophore moieties to become attached to the pathogen particles by the second set of assemblies.

This aspect thus provides a kit of modules that may be assembled around a pathogen particle to effect pathogen fluorescent labelling, followed by fluorophore excitation, fluorescence emission and detection by ancillary detection apparatus.

In a particular aspect of the invention there is provided a method for preparing a virus mimic comprising: selecting a particle having a diameter comparable to that of the virus to be mimicked, and binding a plurality of surface proteins characteristic of the virus to a surface of the particle so as to decorate the surface with the virus surface proteins.

The virus surface proteins should be chosen so as to exhibit selectivity for the first set of macromolecular assemblies on the support surfaces (as hereinbefore described), and for the second set of assemblies to which are attached fluorophore moieties. So for example the sets of assemblies may be aptamers or receptors for the virus mimic surface proteins, preferably in the form of virus spike proteins.

A binding agent may be used to bind the surface proteins to the particle surface. For example if the particle is a polymer micro or nano sphere derivatised with carboxyl/amide, EDC can be used as the binding agent. The binding agent selected will depend upon the nature and material from which the particle is made, subject to any derivatisation of the particle surface.

The surface protein is typically expressed using a plasmid expression vector. It would usually then be purified before binding to the particle surface.

The particle is typically sub-micron in diameter. Most virions are in the range of 5 to 400 nm, so the particles may be in this size range. Particles may be provided by a granular material. In a preferred aspect the particles are synthetic nanospheres or microspheres (also known in the art as beads). For mimicking a SARS-CoV-2 virus the microspheres may be about 100 nm in diameter, which corresponds to that of the virus particle itself. The particle may be a synthetic microsphere or nanosphere. In some embodiments the particle may have a diameter of 10 to 500 nm. In making a multitude of virus mimics a multitude of particles will be used. These should preferably be of substantially uniform size.

The characteristic surface protein may be a spike protein. The surface protein is in a preferred application characteristic of a corona virus, preferably SARS-CoV-2. In the case of a coronavirus the particle may have a diameter of between 80 and 160 nm.

In a related aspect of the invention there is provided a virus mimic comprising: a particle having a diameter comparable to that of the virus being mimicked, the particle having a surface decorated with a plurality of surface proteins characteristic of the virus. The virus mimic may be produced by the method as hereinbefore described.

The virus mimic has the advantage of not being live and therefore can be used more safely in the lab, at lower cost than using procedures and protocols for live virus.

Following is a description by way of example only and with reference to the attached drawings of modes for putting the various aspects of the invention into practice.

In the drawings:

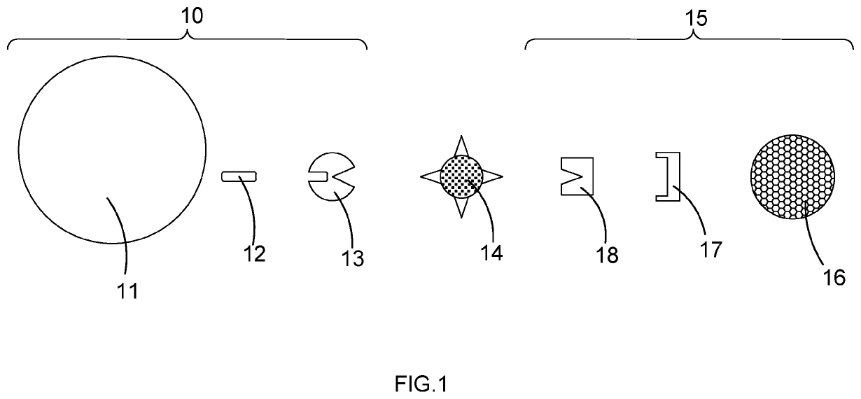

FIG. 1 is a schematic representation of components of a virus capture and signalling system in accordance with the invention.

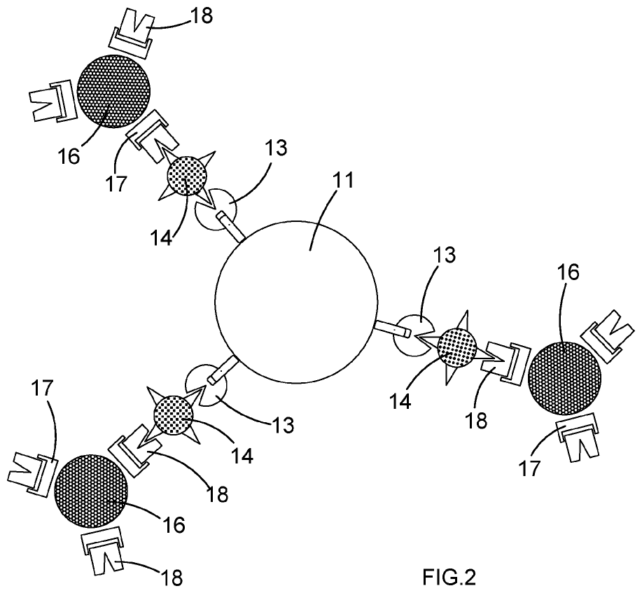

FIG. 2 is a schematic representation of the components of the system assembled to capture virus particles on a microsphere.

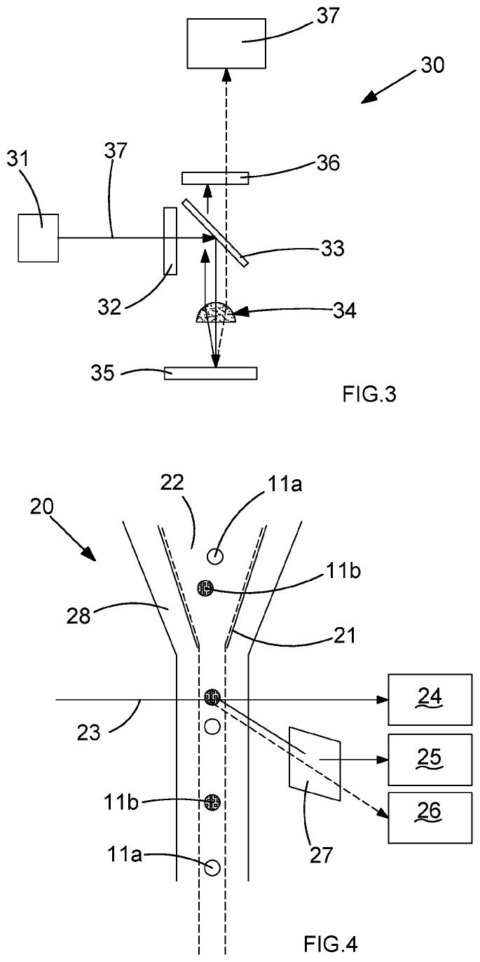

FIG. 3 is a schematic representation of an epifluorescence optical unit of a microscope detection stage.

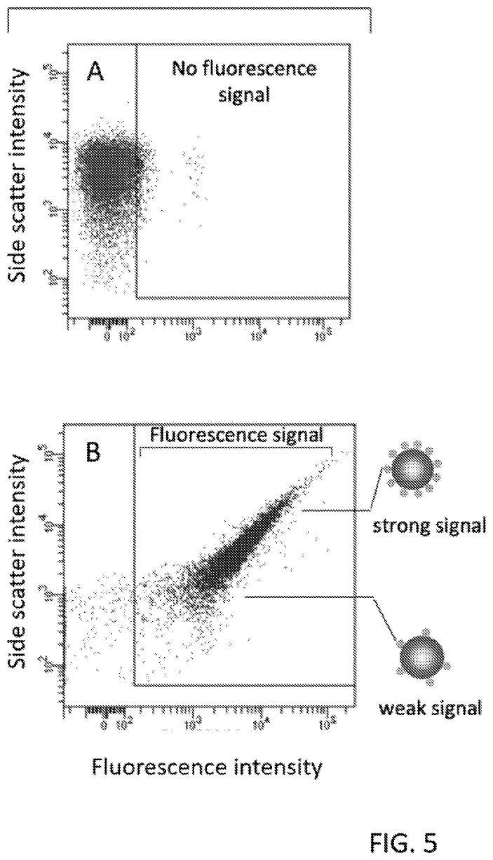

FIG. 4 is a schematic representation of detail of the cytometric detection stage and process.

FIGS. 5A and 5B are graphs showing flow cytometer results for microsphere constructs obtained in the absence of virus mimic particles (FIG. 5A) and in the presence of virus mimic particles (FIG. 5B).

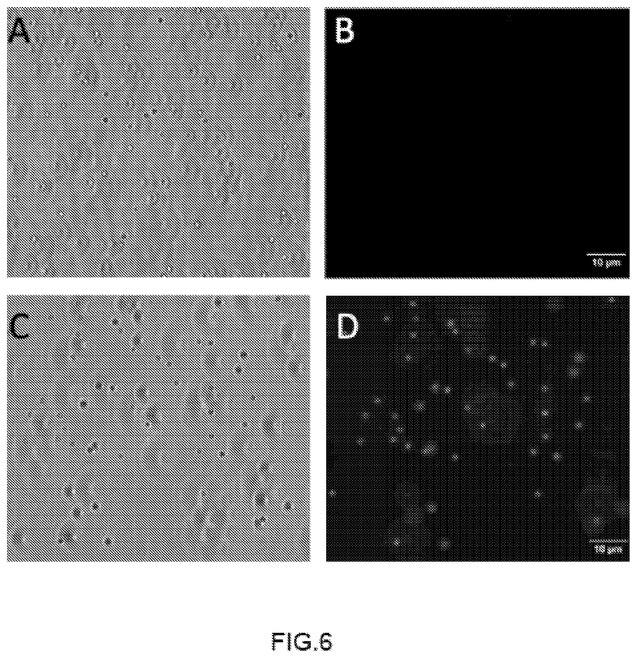

FIG. 6 is a set of four images A, B, C, and D obtained using epifluorescence and bright field microscopy.

FIG. 1 shows schematically components of a pathogen (virus particle) capture module within the span of bracket 10. There is a microsphere 11 having a diameter of 10 microns, and binding protein avidin 12. The avidin binds aptamer 13 to the microsphere surface.

The aptamer binds conformally to the virus particle 14. A signal generation module includes the components within the span of bracket 15. A chimeric protein ACE2-Fc 18 is used to bind to derivatised nanobead 16 via the binding agent Protein G 17. The chimeric protein includes a receptor for the virus particle 14. The assembly obtained after exposure of capture modules and signal modules to virus particles in a test sample is shown in FIG. 2.

In FIG. 2 a single microsphere is shown with three aptamers 13 attached to the sphere surface. In practice there would be a multitude of aptamers distributed over the surface. Each virus particle 14 is shown with four surface spike proteins, one of which is engaged with the aptamer and the other is engaged with the virus receptor portion of the chimeric protein 18. A single nanobead 16 is shown attached to each virus particle, for the sake of clarity. In practice one would expect multiple beads to become attached via multiple spike protein and chimeric protein engagements. Hence a single virus particle may be associated with a plurality of nanobeads, which provides a signal amplification effect. The nanobeads have a larger number of fluorophore molecules, so this provides further signal amplification (as compared to say a virus labelled with a single fluorophore molecule.

Preparation of Derivatised Microspheres for Capturing Virus Particles.

The microspheres are obtained from commercial suppliers (in this case ThermoFisher Scientific Ltd.) in carboxylated or amidated form to facilitate derivatisation. The microspheres are made of latex, are 1 micron in diameter, being of uniform size (with a coefficient of variation in diameter of 4%). These are derivatised with avidin 12 by covalent coupling with 1-Ethyl-3-(3-dimethylaminopropyl) carbodiimide hydrochloride (abbreviation EDC). 10 microns diameter microspheres (coefficient of variation in diameter of 11%) microspheres have also been used successfully.

The microspheres (to provide 1-4% final w/v) are resuspended in either 50 mM MES pH 6.5 or phosphate-buffered saline, PBS, containing 0.1-1% (v/v) Tween-20.

Freshly prepared EDC (50 mg/mL) is added to the microsphere suspension to yield a final EDC concentration of 0.5 to 5 mg/mL. Then avidin is added to the microsphere/EDC mixture to provide a final concentration of 5-100 μg/mL to ensure that the suspension contains three times the manufacturer's calculated surface saturation capacity. The microspheres are incubated either at room temperature for 3 to 4 hr, or overnight at 4° C. with gentle agitation. The microspheres are then centrifuged at 3,000 g to 100,000 g for 5 to 20 min to remove unbound protein. They are washed with PBS containing 0.1-1% Tween-20 before setting them aside for storage in storage buffer (PBS, 0.1% (w/v) glycine, 0.1% (w/v) NaN₃, 0.1-1% (v/v) Tween-20, allowing for glycine to block unbound exposed hydrophobic surfaces.

Preparation of Derivatised Signalling Nanobeads:

The signalling module 15 is shown in FIG. 1b. There are 30 nm diameter fluorescent nanobeads 16, which are coated with Protein G, a guanine nucleotide-binding protein 17. A chimeric protein 18 (ACE-2-Fc) has a portion which binds with Protein G and another portion which binds with virus particles 19. The preparation method is set out below. Carboxylated polystyrene latex fluorescent nanobeads were obtained from ThermoFisher Scientific, sold under the name FluoSpheres™ as carboxylate-modified microspheres. Although nominally described by the manufacturer as being 20 nm diameter, we have found them to be about 30 nm diameter. These are yellow/green fluorescent ‘microspheres’ although better described as nano beads since they are nanoscale (having a dimension less than 100 nm). The beads comprise polystyrene to which has been added fluorescent dye. The 30 nm beads are stated by the manufacturer to provide 180 fluorescein equivalents per nanobead. More generally, synthesis of fluorescent latex nanoparticles (i.e. nanobeads) has been reported by Jacobs et al, in Macromolecules 2014, Vol. 47, iss. 21, 7303-7310.

The nanobeads were derivatised with Protein G by covalent coupling with EDC, by the following method. The nanobeads (1-4% final (w/v)) are suspended in either 50 mM MES buffer pH 6.5 or phosphate-buffered saline, PBS, containing 0.1-1% (v/v) Tween-20. 0.01—Freshly prepared EDC (50 mg/mL) is added to the nanobead suspension to yield a final EDC concentration of 0.5 to 5 mg/mL. Then Protein G is added to the nanobead/EDC mixture to provide a final concentration of 0.75-1.25 mg/mL to ensure that the suspension contains three times the manufacturer's calculated surface saturation capacity. The nanobeads are incubated either at room temperature for 3-4 hr or overnight at 4° C. with gentle agitation.

The nanobeads are then centrifuged at 3,000 g to 100,000 g for 5 to 20 min to remove unbound protein. They are washed with PBS containing 0.1-1% Tween-20 before storing in storage buffer (PBS, 0.1% (w/v) glycine, 0.1% (w/v) NaN₃, 0.1-1% (v/v) Tween-20) allowing for glycine to block unbound exposed hydrophobic surfaces.

Nanobead Functionalisation

ACE2 (angiotensin-converting Enzyme 2) is the host cell receptor to which SARS-CoV-2 binds to cause infection with COVID-19. ACE2-Fc is a chimeric protein which comprises the Fc domain of human IgG. ACE2 binds to the coronavirus S (spike) protein present on the surface of the virion (virus particle). The S protein is a type I protein with four domains that include an S1 (receptor binding subunit), an S2 (membrane-fusion subunit), a transmembrane, and a short intracellular domain. The S protein forms a trimer showing a big protrusion (or spike) from the virus surface.

For the attachment of ACE2-Fc to PrG-derivatised nanobeads, the beads are centrifuged to remove storage buffer and resuspended in PBS+0.1-1% Tween-20, plus three times the calculated equivalent amount of ACE2 required to saturate bound PrG (0.2 mg/mL) on the beads. The beads are then incubated with gentle agitation overnight at 4° C. The beads are then washed with PBS+0.1-1% (v/v) Tween-20, and then stored in a bead storage buffer. The same method can be used to attach ACE2-Fc to microspheres, if desired.

Microsphere Functionalisation

For the attachment of the aptamer to the avidin-derivatised microspheres, we used a single-stranded oligonucleotide containing the specified sequence:

5′ATCCAGAGTGACGCAGCATTTCATCGGGTCCAAAAGGGGCTGCTCGGG
ATTGCGGATATGGACACGT-3′

modified at the 5′ end with biotin triethyleneglycol. This is available from commercial suppliers. In general the microspheres are resuspended in PBS+0.1-1% Tween-20 containing 0.55 mM MgCl₂, plus three times the calculated equivalent amount of aptamer required to saturate bound avidin. The microspheres are incubated with gentle agitation overnight at 4° C. The microspheres are then washed with PBS+0.1-1% Tween-20 containing 0.55 mM MgCl₂, and then stored in microsphere storage buffer, containing 0.55 mM MgCl₂. The same method can be used to attach the aptamer to the nanobeads, if desired.

Sample Test

In practice viral samples will be obtained from throat swabs of infected patients. In the proof of concept tests described in the present application an engineered viral mimic is used instead. This is a microsphere particle which is decorated with SARS-Cov2 spikes. These are added to the bead storage buffer to provide a ‘virus’ load. The preparation of the viral mimic for COVID-19 is described below.

Functionalised nanobeads and microspheres are resuspended in either 1 mL bead storage buffer (+/−0.55 mM MgCl₂), PBS+0.1% Tween-20 or 50 mM MES+0.1% Tween-20 and gently agitated for up to 2 hr at ambient temperature, or overnight.

To detect SARS-Cov2-spike bearing particles, about 100,000 capture module microspheres are exposed to the virus-containing solution so that the virus particles (more correctly termed virions) become bound to the aptamers on the surface of the microspheres, by means of the virus spike conformally binding to the counterpart aptamer on the microsphere. 100,000 is a representative value within the target range for detection of COVID-19.

The microspheres loaded with virus are then incubated with saturating numbers (4×10⁸-4×10¹⁰) of functionalised signalling nanobeads. Each virion has a diameter of around 100 nm and has about 60 protein spikes. Not all of these spikes will be spatially accessible, but nevertheless a large number of nanobeads can bind to each virion.

As shown schematically in FIG. 2, the liquid sample now includes microsphere constructs each comprising a microsphere 11 covered by a multitude of captured virions 14 (three only shown), with each virion provided with a plurality of fluorescent nanobeads 16 (only one bead per virion shown). It will be seen that each microsphere can be loaded with a multitude of captured or anchored virions, and each virion carries a multitude of signalling nanobeads (each of which contains the equivalent of about 180 fluorescein molecules in this embodiment). Hence the potential fluorescence signal for a virion is amplified as a function of the number of nanobeads and the number of fluorescein molecules per nanobead.

Microsphere Detection—Fluorescence Microscopy

In FIG. 3 a basic fluorescence detection system 30 is shown. The system includes an excitation filter 32 for admitting excitation light 37 from a laser or LED source 31, a dichroic filter 33, a microscope objective lens 34, a microscope stage 35 on which the objective lens is focused and containing test sample microbeads immobilised in a mountant high viscosity liquid (not visible). There is an emission filter 36 through which emitted light (dashed line) passes to a digital sensor (camera) 37 for detecting, imaging and recording fluorescence from microspheres in the test sample in the stage. The fluorescence can instead be observed visually by a person, so as to give a quick and definitive detection confirmation.

Microsphere Detection—Flow Cytometry

The liquid suspension of microspheres is fed into the nozzle of a flow cytometer, 20, such as is shown in FIG. 4. The liquid sample 22 with entrained microspheres 11a and fluorescing microspheres 11b is fed through a frustoconical nozzle 21, designed in conjunction with flow of sheath fluid 28 to ensure that microspheres pass sequentially therethrough. A focused laser beam 23 is used to excite fluorophores as the fluorescent microspheres pass through the beam. The fluorescent light emitted orthogonal to the excitation beam is detected by a detector 26, and light scattering is concurrently monitored by a forward scattering (FSC) detector 24 and/or a side scattering (SSC) detector 25 after separation from fluorescent light using a dichroic mirror 27 and optical bandpass filters, as are known in the art. In this embodiment the FSC detector 24 was a photodiode, and photomultipliers were used for SSC detection and fluorescence detection. Individual microspheres can be resolved by the method, and intensity of light emitted by each microsphere measured, and if desired integrated.

In the present example, analysis was undertaken using a commercially available Becton Dickinson LSRII Flow Cytometer, using 488 nm laser illumination, with data collected and analysed using Becton Dickinson FACSDiva 8.0.1 software. Each microsphere construct passing sequentially through the nozzle 21 can be resolved by the focused imaging beam. The fluorescence emitted by each microsphere confirms the presence of virions, and the intensity of fluorescence allows an estimate of the viral loading of each microsphere to be made. These signals are recorded to derive an implied accumulated viral loading for the test swab. For proof of concept the virus mimic particles were used instead of live virus. The production of the virus mimic is described in more detail hereinafter.

FIG. 5 shows output scatter graphs from two sets of cytometry runs labelled A and B conducted on microsphere constructs in accordance with the invention. Capture modules (functionalised microspheres 1 micron in diameter, as hereinbefore described) and signal modules (functionalised nanobeads as hereinbefore described) were mixed with virus mimic particles and incubated. The liquid suspension of microsphere constructs thereby obtained was enriched for the microsphere constructs by centrifugation. The pelleted microsphere constructs obtained were then re-suspended in bead storage buffer. Flow cytometry was conducted on these microsphere constructs to produce the fluorescence data shown in FIG. 5B. The procedure was repeated using capture modules and signal modules, but this time incubated in the absence of virus mimic particles. Cytometry of the microsphere constructs obtained in this procedure produced the data shown in FIG. 5A.

In these data sets the Y axis shows side scatter intensity (which is a measure of microsphere construct size). The X axis indicates fluorescence intensity from each microsphere construct. The second dataset in FIG. 5B is consistent with the functionalised microspheres capturing virus mimic particles and with nanobeads also becoming attached to virus mimic particles. The more nanobeads are attached to each virus mimic particle, and the more virus mimic particles are attached to each microsphere, the higher the fluorescence signal intensity. A strong fluorescence signal is thus indicative of a large number of nanobeads in each microsphere construct, and a weaker signal indicates fewer nanobeads. By comparison, the first dataset shown in FIG. 5A has an essentially non-existent fluorescence signal. This is consistent with the functionalised nanobeads only being able to co-locate with the functionalised microspheres when the virus mimic particles are also present. Hence the microsphere constructs subjected to cytometry would be expected to be simply the initial fluorophore-free functionalised microspheres. What little fluorescence signal can be detected is likely due to small numbers of nanobeads remaining in the pelleted material after the centrifugation.

In FIG. 6 there is a set of images obtained using epifluorescence microscopy of samples of the type discussed above in relation to FIG. 5 used in flow cytometry. In this case the pelleted microsphere constructs are re-suspended in a mountant liquid and spread on respective microscope slides. The mountant renders the microsphere constructs immobile. The images in A&B and C&D of FIG. 6 have the same respective fields of view. Images A and C are brightfield images and show microsphere constructs as dots in each image. Images B and D used fluorescence illumination (excitation wavelength 480 nm, emission wavelength 517 nm). The microspheres visible in image A have no signal modules attached thereto and thus are invisible in fluorescence image B, which is black. The microspheres in image C have signal modules attached, with their associated fluorescent nanobeads. These thus fluoresce as shown in image D (they appear grey in the monochrome image but are in practice fluorescent green).

Production and Engineering of COVID-19 Virus Mimic.

An engineered virus mimic was used in the example described above for reasons of laboratory safety and convenience. The virus mimic comprises microspheres with a diameter comparable with that of the mimicked virus particle. These microspheres are surface-decorated with engineered surface proteins (the virus spike protein). In the present case the virus mimic of SARS-CoV-2 was created using the following steps.

Expression of coronaviral surface protein (spike). HEK 293F suspension cells (commercially available from ThermoFisher) were seeded at a density of 6×10\hat{ }5 cells/mL in Expi293 expression medium (also from ThermoFisher). These were incubated in an orbital shaking incubator at 37° C. and 125 RPM with 8% CO₂ and allowed to grow to cell density no greater than 5×10\hat{ }6 cells/mL before passaging. For transfection, about 600×10\hat{ }6 cells were suspended in 200 mL of Expi293 expression media in a 1 L conical tissue culture flask. A mammalian expression plasmid (200 mg) enabling the expression of a His-tagged SARS-Cov2 spike protein was added to 12 mL Opti-MEM (from ThermoFisher) cell culture medium. To this is added another 12 mL Opti-MEM medium to which 640 mL of ExpiFectamine transfection reagent had previously been added. After 10 min incubation at ambient temperature, the mixture was added drop-wise to the cell suspension, which was subsequently returned to an orbital shaking incubator at 37° C. and 125 RPM with 8% CO₂. After 16-18 h, 1.2 mL of Expifectamine 293 Transfection Enhancer 1 and 12.1 mL of Expifectamine 293 Transfection Enhancer 2 was added to the culture. Three days after transfection, the cell suspension was harvested, and cells and cell debris were removed from the medium by centrifugation at 4,000 g for 20 min at 4° C. The supernatant was retained and filtered using a 0.22 μm sterile filter, and stored at 4° C. until required for purification.

Purification of Coronavirus Surface Protein (Spike)

Prior to use, Ni-NTA resin (6 mL) was washed with phosphate buffered saline, then centrifuged at 1000 g for 10 min. The resin was retained, and the cell supernatant added with gentle shaking. The resin was incubated with the supernatant for 1 h on a rotating shaker at 4° C. A polypropylene column was loaded with the supernatant-resin mixture and then washed with a minimum of 4 column volumes of 50 mM sodium phosphate, 300 mM NaCl, 20 mM imidazole, pH 8.0. Protein elution was obtained using 50 mM sodium phosphate, 300 mM NaCl, 250 mM imidazole, pH 8.0. Protein-containing fractions were combined, concentrated and buffer exchanged using a centrifugal protein concentrator, into phosphate buffered saline.

Decorating Microspheres with Spikes

Carboxylated/amidated latex microspheres (100 nm in diameter) were resuspended to 4% (w/v) in 50 mM MES buffer pH 6.5 with Tween-20 concentrations varying between 0.1-1%. 50 mg/mL fresh EDC was added to the microspheres at a ratio of 1/100 of buffer volume. Spike protein was added to the microsphere/EDC mixture to provide a surface coating of ˜25-100 molecules per microsphere. The microspheres were incubated either at room temperature for 3-4 hr or overnight at 4° C. with gentle agitation to generate the microsphere-based virus mimics. These microspheres were then centrifuged at 30,000 g for 20 min to remove unbound protein, and washed with PBS containing 0.1-1% Tween-20. The microspheres are then stored in storage buffer (PBS, 0.1% glycine, 0.1% NaN₃, 0.1-1% Tween-20) allowing for glycine to block unbound reactive sites.

The decorated microspheres were used as virus particle mimics in the methods described above.

In summary, the present invention concerns the field of diagnostic testing to confirm the presence of target pathogens in a test sample, typically obtained from a human or animal. The test has particular application in testing for viruses, but could include cancer cells or other particulate pathogens, including bacteria. In a core aspect the disclosure provides a method for facilitating the detection of target pathogen particles in a test sample, the method comprising: providing one or more supports defining a support surface, the support surface being provided with a coating of a first set of macromolecular assemblies, the assemblies each being capable of selectively binding with the target pathogen particles, providing a second set of macromolecular assemblies, the assemblies also each being capable of binding selectively with the target pathogen particles, and wherein each of the second set of macromolecular assemblies is provided with at least one fluorophore moiety, obtaining or providing the test sample to be assayed for the presence of the target pathogen therein, exposing the macromolecular assemblies to the test sample so that target pathogen particles present in the test sample bind to the macromolecular assemblies, thereby producing a multitude of target pathogen particles distributed over and anchored to said support surface by members of the first set of macromolecular assemblies, with a plurality of fluorophore moieties being bound to each pathogen particle by members of the second set of macromolecular assemblies, so as to produce a fluorophore coating on anchored pathogen particles on the support. The invention provides assemblies of pathogen particles produced by this method. The methods may include a detection step for detecting fluorescence from bound fluorophores.

1. A method for facilitating the detection of target pathogen particles in a test sample, the method comprising:
providing one or more support defining a support surface,
the support surface being provided with a coating of a first set of macromolecular assemblies, the assemblies each being capable of selectively binding with the target pathogen particles,
providing a second set of macromolecular assemblies, the assemblies also each being capable of binding selectively with the target pathogen particles, and wherein each of the second set of macromolecular assemblies is provided with at least one fluorophore moiety,
obtaining or providing the test sample to be assayed for the presence of the target pathogen therein,
exposing the macromolecular assemblies to the test sample so that target pathogen particles present in the test sample bind to the macromolecular assemblies, thereby producing a multitude of target pathogen particles distributed over and anchored to said support surface by members of the first set of macromolecular assemblies, with a plurality of fluorophore moieties being bound to each pathogen particle by members of the second set of macromolecular assemblies, so as to produce a fluorophore coating on anchored pathogen particles on the support.

2. A method as claimed in claim 1 and sequenced so that the first set of macromolecular assemblies is exposed to the test sample before the second set of macromolecular assemblies is exposed thereto, so that the anchorage of the pathogen particles to the support takes place substantially before the binding of the fluorophore moieties to the pathogen particles.

3. A method as claimed in claim 1 or claim 2 wherein said at least one support comprises one or more selected from: a plurality of support particles, or a plurality of fibres, or a textile or felt material, or a mesh, or a grid, or a frit, or a chromatography monolith, or a filter material or other porous membrane.

4. A diagnostic method for detecting target pathogen particles in a test sample comprising conducting the method of any of claims 1 to 3 and further comprising exposing the fluorophore-coated support to incident light radiation so as to cause excitation of the fluorophores and emission of fluorescence.

5. A method as claimed in claim 4 further comprising detecting the emitted fluorescence so as to indicate the presence of pathogen particles in the test sample.

6. A method as claimed in claim 4 or claim 5 wherein the fluorescence is detected by fluorescence microscopy.

7. A method as claimed in any of claims 4 to 6 wherein the support comprises a plurality of support particles and flow cytometric apparatus is provided in which support particles are induced to flow sequentially through a focused excitation source, and emitted fluorescence associated with each support particle is detected.

8. A method as claimed in claim 7 wherein the cytometric apparatus is adapted to determine the intensity of fluorescence associated with each support particle.

9. A method as claimed in any of claims 5 to 8 wherein the emissions detected are integrated.

10. A method as claimed in any of the preceding claims wherein the test sample is a liquid containing pathogen particles dispersed therein.

11. A method as claimed in claim 10 wherein the test sample comprises an aerosol of liquid droplets each having pathogen particles dispersed therein.

12. A method as claimed in claim 10 or 11 wherein said support is arranged to be immobile when exposed to the test sample.

13. A method as claimed in claim 12 wherein said support is immobile with respect to a flow of test sample over the support so as to expose the support to pathogen particles.

14. A method as claimed in any of the preceding claims wherein the support comprises support particles coated with the first set of assemblies.

15. A method as claimed in claim 14 wherein the support particles are arranged as a bed of particles, or a packed conglomeration of particles.

16. A method as claimed in any of claim 14 or 15 wherein a fluid comprising the test sample is introduced to the support particles, so that any pathogen particles present bind to the support particles.

17. A method as claimed in any of claims 14 to 16 wherein a liquid comprising the second set of assemblies comprising fluorophores is introduced to the support particles so that fluorophores become attached to pathogen particles on the support particles by means of the second set of assemblies.

18. A method as claimed in any of the preceding claims wherein the support comprises support particles comprising microspheres, or a granular material.

19. A method as claimed in claim any preceding claim wherein the support particles have a diameter of about 0.5 to 100 microns, preferably about 0.5 to 20 microns.

20. A method as claimed in any of the preceding claims wherein a plurality of pathogen particles become bound to each support, and a multitude of fluorophore moieties are bound to each pathogen particle.

21. A method as claimed in any of the preceding claims wherein each fluorophore moiety comprises a fluorescent nanobead.

22. A method as claimed in claim 21 wherein each nanobead comprises a multitude of fluorophore molecules.

23. A method as claimed any of the preceding claims wherein the macromolecular assemblies each comprise proteins and/or nucleic acids.

24. A method as claimed in any of the preceding claims wherein the macromolecular assemblies comprise an aptamer which selectively binds to the pathogen, or a receptor for the pathogen.

25. A method as claimed in claim 24 when one set of macromolecular assemblies comprises aptamers and the other set comprises receptors for the target pathogen.

26. A method as claimed in any of the preceding claims wherein the second set of macromolecular assemblies each comprise a chimeric protein, one portion of which is adapted to bind selectively to the pathogen and the other portion of which is bound to the fluorophore moiety.

27. A method as claimed in any of the preceding claims wherein the pathogen is a virion, a portion of a virion, a bacterium, or a cancer cell.

28. A method as claimed in any of the preceding claims wherein the pathogen is a virion, preferably a corona virion, most preferably SARS-CoV-2.

29. A method as claimed in claim 28 wherein the second set of macromolecular assemblies comprises a chimeric protein and one portion of the chimeric protein is selected from an aptamer for the virion, or a receptor for the virion, and the other is bound to the fluorophore moiety.

30. A method as claimed in any of claims 27 to 29 wherein each virion is bound to a support particle by an aptamer which binds conformally with the virion, or by a receptor which binds with a surface protein of the virus.

31. A method as claimed in any of claims 28 to 30 wherein one portion of the chimeric protein comprises angiotensin-converting enzyme 2 (ACE2) which serves as a receptor for the coronavirus.

32. A method as claimed in any of claims 24 to 31 wherein the fluorophore moiety bound to each chimeric protein comprises one or more fluorescent nanobead.

33. A method as claimed in any of the preceding claims wherein one or more binding agents are used to promote or effect binding between the support surface and the first set of macromolecular assemblies.

34. A method as claimed any or the preceding claims wherein one or more binding agents are used to promote or effect binding between the fluorophore moieties and the second set of macromolecular assemblies.

35. A fluorescent assembly comprising a support with a plurality of pathogen particles anchored to the support by a first set of macromolecular assemblies, and a plurality of fluorophore moieties attached to each pathogen particle by a second set of macromolecular assemblies.

36. A fluorescent assembly in accordance with claim 35 obtainable by the method of any of the preceding claims.

37. An assembly of pathogen particles comprising:
a support defining a support surface,
a first set of macromolecular assemblies each having one region bound to the support surface and another region bound to a target pathogen particle, so that there is a multitude of anchored target pathogen particles on the support surface,
a second set of macromolecular assemblies each having one region bound to the pathogen particles and another region with a fluorophore moiety attached thereto.

38. An assembly as claimed in claim 37 wherein each support has multiple pathogen particles attached thereto, and multiple fluorophore moieties attached to each pathogen particle.

39. An assembly as claimed in claim 37 or 38 wherein the fluorophore moieties comprise fluorescent nanobeads each comprising multiple fluorescent molecules.

40. A target pathogen particle labelling system comprising:
(i) a capture module comprising a support defining a support surface, and a first set of macromolecular assemblies each having one region bound to the support surface, and another region which is adapted to selectively bind with the target pathogen particle; and
(ii) a signal module comprising a second set of macromolecular assemblies each having one region adapted to selectively bind to pathogen particles, and another region with a fluorophore moiety attached thereto,
wherein a test sample containing comprising pathogen particles may be exposed to multiple capture modules and multiple signal modules so as to cause multiple pathogen particles to become bound to the support by the first set of assemblies, and multiple fluorophore moieties to become attached to the pathogen particles by the second set of assemblies.

41. A method for preparing a virus mimic comprising:
selecting a particle having a diameter comparable to that of the virus to be mimicked, and
binding a plurality of surface proteins characteristic of the virus to a surface of the particle so as to decorate the surface with the virus surface proteins.

42. A method as claimed in claim 41 wherein a binding agent is used to bind the surface proteins to the particle surface.

43. A method as claimed in any of the preceding claims wherein the surface protein is expressed using a plasmid expression vector and then purified before binding to the particle surface.

44. A method as claimed in any of claims 41 to 43 wherein the particle is sub-micron in diameter.

45. A method as claimed in any of claims 41 to 44 wherein the particle is a synthetic microsphere or nanosphere.

46. A method as claimed in any of claims 41 to 45 wherein the particle has a diameter of 10 to 500 nm.

47. A method as claimed in any of claims 41 to 46 wherein the surface protein is a spike protein.

48. A method as claimed in any of claims 41 to 47 wherein the surface protein is characteristic of a corona virus, preferably SARS-CoV-2.

49. A method as claimed in claim 48 wherein the particle has a diameter of between 80 and 160 nm.

50. A virus mimic comprising: a particle having a diameter comparable to that of the virus being mimicked, the particle having a surface decorated with a plurality of surface proteins characteristic of the virus.

51. A virus mimic obtainable by the method of any of claims 41 to 49.


SEQUENCE LISTING (1 sequence)

SEQ ID NO: 1 - DNA, 67 bp, Artificial Sequence
  Synthetic construct
atccagagtgacgcagcatttcatcgggtccaaaaggggctgctcgggattgcggatatggacacgt